SEQUENCE LISTING (21 sequences)

SEQ ID NO: 1 - DNA, 19 bp, Artificial Sequence
  T7 promoter
taatacgactcactatagg

SEQ ID NO: 2 - DNA, 19 bp, Artificial Sequence
  T3 promoter
aattaaccctcactaaagg

SEQ ID NO: 3 - DNA, 18 bp, Artificial Sequence
  sP6 promoter
atttaggtgacactatag

SEQ ID NO: 4 - DNA, 22 bp, Artificial Sequence
  A1
ctaaattaaccctcactaaagg

SEQ ID NO: 5 - DNA, 19 bp, Artificial Sequence
  T3gRNA
ctaaattaaccctcactaa

SEQ ID NO: 6 - DNA, 22 bp, Artificial Sequence
  B1/C1
cgcaattaaccctcactaaagg

SEQ ID NO: 7 - DNA, 25 bp, Artificial Sequence
  mm
cctttctcgtgaacgcgtcttcagg

SEQ ID NO: 8 - DNA, 20 bp, Artificial Sequence
  mLAgRNA
cctttctcgtgaactagtca

SEQ ID NO: 9 - DNA, 23 bp, Artificial Sequence
  m
cctttctcgtgaactagtcatgg

SEQ ID NO: 10 - DNA, 25 bp, Artificial Sequence
  mLAsgF
caccgcctttctcgtgaactagtca

SEQ ID NO: 11 - DNA, 25 bp, Artificial Sequence
  mLAsgR
aaactgactagttcacgagaaaggc

SEQ ID NO: 12 - DNA, 41 bp, Artificial Sequence
  sgLAF
tgtaatacgactcactataggcctttctcgtgaactagtca

SEQ ID NO: 13 - DNA, 20 bp, Artificial Sequence
  sgRNAR
aaaagcaccgactcggtgcc

SEQ ID NO: 14 - DNA, 60 bp, Artificial Sequence
  T3gRNAF
tgtaatacgactcactataggctaaattaaccctcactaagttttagagctagaaatagc

SEQ ID NO: 15 - DNA, 57 bp, Artificial Sequence
  AqugblockF
gctgtctcatcattttggcaaagaattcgaacttagtcatggtgtcaaagggtgagg

SEQ ID NO: 16 - DNA, 12 bp, Artificial Sequence
  AqugblockR
ttggtggccggg

SEQ ID NO: 17 - DNA, 40 bp, Artificial Sequence
  nucleotides of the of the right homologous sequence of a gBlock fragment for DNA assembly
ttggtggccgggaaggggcacgattgtcttcactagccat

SEQ ID NO: 18 - DNA, 22 bp, Artificial Sequence
  FRT partial
cctattctctagaaagtatagg

SEQ ID NO: 19 - DNA, 19 bp, Artificial Sequence
  FrtsgRNA
cctattctctagaaagtat

SEQ ID NO: 20 - DNA, 22 bp, Artificial Sequence
  loxP(partial)
tcgtataatgtatgctatacga

SEQ ID NO: 21 - DNA, 19 bp, Artificial Sequence
  loxPsgRNA
tcgtataatgtatgctata

(19) United States
(12) Patent Application Publication
(10) Pub. No.: US 2018/0002706 A1
(43) Pub. Date: Jan. 4, 2018

(54) METHODS AND COMPOSITIONS FOR CLONING INTO LARGE VECTORS

(71) Applicant: UNIVERSITY OF SOUTH FLORIDA, Tampa, FL (US)

(72) Inventors: JIA-WANG WANG, TAMPA, FL (US); RICHARD F. LOCKEY, TAMPA, FL (US); KUNYU LI, TAMPA, FL (US)

(73) Assignee: University of South Florida, Tampa, FL (US)

(21) Appl. No.: 15/541,116

(22) PCT Filed: Dec. 18, 2015
(86) PCT No.: PCT/US15/66732

§ 371 Date: Jun. 30, 2017

Related U.S. Application Data
(60) Provisional application No. 62/097,770, filed on Dec. 30, 2014.

(51) Int. Cl. C12N 15/66; C12N 15/10

(52) U.S. Cl. CPC C12N 15/66; C12N 15/1031

(57) ABSTRACT

Provided herein are methods of cloning into vectors.

### CROSS-REFERENCE TO RELATED APPLICATIONS

This application claims the benefit of U.S. Provisional Application Ser. No. 62/097,770 filed on Dec. 30, 2014, having the title “Methods and Compositions for Cloning into Large Vectors” the entirety of which is incorporated herein by reference.

### SEQUENCE LISTING

This application contains a sequence listing filed in electronic form as an ASCII.txt file entitled 02326437.txt, created on Dec. 18, 2015, and having a size of 4,124 bytes. The content of the sequence listing is incorporated herein in its entirety.

### BACKGROUND

Cloning is an essential tool for genetic engineering. Many cloning techniques have been developed and most rely on cleaving DNA via restriction enzymes. Restriction enzymes have some characteristics, which set limitations on their use in cloning. For example, restriction enzymes cannot cleave at any given location in a sequence. Additionally, restriction enzymes may have multiple cleavages sites within a DNA segment or vector of interest. Despite several new cloning strategies having been developed over time, these methods still do not provide for easy and efficient cloning into larger vectors. Therefore, there exists an unmet need for improved tools and strategies for cloning, especially into larger vectors.

### SUMMARY

In some embodiments, the methods provided herein can including the steps of synthesizing an sgRNA that can have a crRNA sequence operatively linked to a tracRNA sequence, where the crRNA sequence can be complementary to a target sequence in a substrate vector, incubating the sgRNA with an amount of a Cas9 endonuclease and an amount of the substrate vector to produce a linearized cleaved substrate vector having a cleavage point and incubating an amount of linearized cleaved substrate vector with an amount of an insert polynucleotide and an amount of at least one of the following: a DNA ligase, a DNA exonuclease a DNA polymerase, or a combination thereof, where the insert polynucleotide comprises a 5′ end sequence that is complementary with a first polynucleotide sequence in the substrate vector and a 3′ end sequence that can be complementary with a second polynucleotide sequence in the substrate vector, and where the first polynucleotide sequence and the second polynucleotide sequence can be on opposite sides of the cleavage point. In some embodiments, the sgRNA can also be incubated with an amount of a suitable single stranded DNA binding protein with the amount Cas9 endonuclease during the step of incubating the sgRNA with the amount of a Cas9 endonuclease and the amount of the substrate vector to produce the linearized cleaved substrate vector with the cleavage point. In embodiments, the suitable single stranded DNA binding protein can be at least one of Tth RecA, a helicase, a single stranded DNA binding protein, or E. coli RecA. In embodiments, the sgRNA can also be incubated with an amount of a adenosine triphosphate with the amount Cas9 endonuclease and single stranded DNA binding protein during the step of incubating the sgRNA with the amount of a Cas9 endonuclease and the amount of the substrate vector to produce the linearized cleaved substrate vector with the cleavage point.

In embodiments, the step of synthesizing sgRNA can include the steps of: performing a polymerase chain reaction (PCR) to produce a duplex DNA template, wherein the PCR reaction contains an amount of a template DNA, an amount of a forward primer, and an amount of a reverse primer, where the forward primer can include: a polynucleotide sequence that can bind a RNA polymerase; a CRISPR-related RNA (crRNA) polynucleotide, where the crRNA polynucleotide can be operatively linked to the polynucleotide sequence that can bind a RNA polymerase and a tracrRNA polynucleotide, where the tracrRNA polynucleotide can be operatively linked to the crRNA polynucleotide and operatively linked to the polynucleotide sequence that can bind a RNA polymerase; and performing in vitro transcription on the duplex DNA template to produce the sgRNA. In embodiments, the RNA polymerase is T3, T7, or sP6.

In embodiments, the target sequence in the substrate vector can be adjacent to a protospacer adjacent motif sequence in the substrate vector. In embodiments, the tracrRNA polynucleotide can be 20 base pairs in length. In embodiments, the crRNA polynucleotide is 19 base pairs.

In some embodiments, the first polynucleotide sequence in the substrate vector and the second polynucleotide sequence in the substrate vector can be about 20 to about 40 base pairs in length. In embodiments, the ratio of linearized cleaved substrate vector to polynucleotide insert can range from about 1:1 to about 1:10 to about 10:1. In some embodiments, the step of incubating an amount of linearized cleaved substrate vector can be conducted at about 35° C. to about 50° C. In embodiments, the substrate vector can be a large vector. In embodiments, the substrate vector can be about 2 kb to about 2 Mb. In embodiments, the substrate vector can be a yeast artificial chromosome, bacterial artificial chromosome, adenoviral vector, cosmid, or baculoviral vector.

In some embodiments, the method can include the steps of synthesizing an sgRNA having a crRNA sequence operatively linked to a tracRNA sequence, where the crRNA sequence is complementary to a target sequence in substrate genomic DNA and incubating the sgRNA with an amount of a Cas9 endonuclease, an amount of a suitable single stranded binding protein, and an amount of substrate genomic DNA to produce a cleaved substrate genomic DNA having a cleavage point. In embodiments, the method can include the step of incubating an amount of cleaved substrate genomic DNA with an amount of an insert polynucleotide, an amount of an exonuclease, an amount of a DNA polymerase, and an amount of a DNA ligase, where the insert polynucleotide contains a 5′ end sequence that can be complementary with a first polynucleotide sequence in the cleaved substrate genomic DNA and a 3′ end sequence that is complementary with a second polynucleotide sequence in the cleaved substrate genomic DNA, and where the first polynucleotide sequence and the second polynucleotide sequence can be on opposite sides of the cleavage point. In some embodiments, the suitable single stranded DNA binding protein is at least one of Tth RecA, a helicase, Extreme Thermostable single stranded DNA binding protein, E. coli RecA. In embodiments, the sgRNA can also be incubated with an amount of a adenosine triphosphate with the amount Cas9 endonuclease and single stranded DNA binding protein during the step of incubating the sgRNA with the amount of a Cas9 endonuclease and the amount of the substrate genomic DNA to produce the cleaved substrate genomic DNA having the cleavage point.

In some embodiments, the step of synthesizing sgRNA includes the steps of: performing a polymerase chain reaction (PCR) to produce a duplex DNA template, wherein the PCR reaction contains an amount of a template DNA, an amount of a forward primer, and an amount of a reverse primer, where the forward primer contains a polynucleotide sequence that can bind a RNA polymerase, a CRISPR-related RNA (crRNA) polynucleotide, where the crRNA polynucleotide is operatively linked to the polynucleotide sequence that can bind a RNA polymerase, and a tracrRNA polynucleotide, where the tracrRNA polynucleotide is operatively linked to the crRNA polynucleotide and operatively linked to the polynucleotide sequence that can bind a RNA polymerase, and performing in vitro transcription on the duplex DNA template to produce the sgRNA. In embodiments, the RNA polymerase can be T3, T7, or sP6. In embodiments, the target sequence in the substrate genomic DNA is adjacent to a protospacer adjacent motif (PAM) sequence in the substrate vector. In embodiments, the target sequence in the substrate genomic DNA is not adjacent to a protospace adjacent motif (PAM) sequence in the substrate vector. In some embodiments, the tracrRNA polynucleotide can be 80 bases in length. In some embodiments, the crRNA polynucleotide is 19 bases. In some embodiments, the first polynucleotide sequence in the substrate genomic DNA and the second polynucleotide sequence in the substrate genomic DNA can be about 17 to about 40 base pairs in length. In embodiments, the ratio of linearized cleaved substrate genomic DNA to polynucleotide insert can range from about 1:1 to about 1:10 to about 10:1. In some embodiments, the step of incubating an amount of cleaved substrate genomic DNA can be conducted at about 35° C. to about 50° C. In some embodiments, the substrate genomic DNA is non-human.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 shows one embodiment of a method of cloning into a large vector.

FIG. 2 shows another embodiment of a method of cloning into a large vector.

FIGS. 3A-3E demonstrates crRNA size on Cas9/sgRNA digestion (3A). Plasmid A1 is a 22 kilobase (kb) target vector and has the 19 base pair (bp) crRNA (T3gRNA) binding sequence while plasmid B1 & C1 have a 16 bp crRNA binding sequence. Arrows indicate the expected Cas9 digested band for each plasmid when cut with Pvu1, The X denotes the band un-cleaved by the Cas9/T3gRNA. FIG. 3B demonstrates Cas9/sgRNA digestion requires the presence of the sgRNA sequences. The three positive clones (G5-7) and two negative clones (Q1-2) obtained from the CRISPR/Gibson cloning were digested with the Cas9/T3gRNA. A positive clone has the insert but does not have the crRNA, while a negative clone does not have the insert but does have the crRNA sequence. As demonstrated in FIG. 3C, Cas9/sgRNA does not cleave the sequence with high homology with the crRNA sequence. The vector E was modified from the vector D. The D vector has the crRNA sequence (m, match), while the E vector does not have the crRNA sequence but a sequence that has several mismatches (mm) with the crRNA sequence. FIG. 3D demonstrates the sequence alignment of the two sequences from plasmid A1 and plasmid B1 & C1 with the 19 bp T3gRNA sequence. FIG. 3E demonstrates the sequence alignment of the two sequences from the vectors D and E that are matched (m) and mismatched (mm), respectively, with the crRNA sequence. The PAMs including the 5′-NAG are also shown as underlined. The number is the length of the corresponding sequence shown in FIGS. 3D and 3E.

FIGS. 4A-4C demonstrates the results after Gibson cloning. FIG. 4A demonstrates restriction enzyme characterization of plasmid DNA extracted from 4 clones from Gibson cloning and 4 clones from QC cloning. All the clones shown were double digested with NheI/PspXI. A PspXI site is present in the insert but not in the vector. Clone Q1-4 from QC cloning are negative. Clone G5-8 from Gibson cloning were positive indicated by the presence of the smaller top band and the bottom double bands. FIG. 4B demonstrates the sequencing chromatograms showing that the insert is correctly cloned into the vector at one bp accuracy at the 5′ end. The shadowed sequence is the homologous sequence in the forward PCR primer. FIG. 4C demonstrates the sequencing chromatograms showing that the insert is correctly cloned into the vector at one bp accuracy at the 3′ end. The shadowed sequence is the reverse primer, which was part of the homologous sequence used in Gibson assembly, used to amplify the insert.

FIG. 5 shows a table demonstrating the predicted plasmid DNA fragments digested by restriction enzyme and Cas9/sgRNA. The plasmid DNA fragments were predicting using the NEBcleaveter version 2.0 online software available from New England Biolabs.

FIGS. 6A and 6B demonstrate that Cas9 may have topoisomerase activity that can change plasmid conformation. As shown in FIG. 6A, Cas9/sgRNA digestion requires the presence of the crRNA sequences. The three positive clones (Nos. 5, 6 and 7) and two negative clones (Nos. 1 and 2) obtained from the CRISPR/Gibson cloning were digested with the Cas9/T3gRNA. A positive clone has the insert and the crRNA sequence has been deleted, while a negative clone does not have the insert but the crRNA sequence.crRNA crRNA. As shown in FIG. 6B, Cas9/sgRNA does not cleave the sequence with high homology with the crRNA sequence. The vector E was modified from the vector D. The D vector has the crRNA sequence (m, match), while the E vector does not have the crRNA sequence but a sequence that has several mismatches (mm) with the crRNA sequence.crRNA crRNA

FIG. 7 shows a table demonstrating the nucleotide sequences referenced herein. *crRNA sequences are in bold.

FIG. 8 shows a plasmid map of pLACAGRFP/tetonAqua and demonstrates the positions of the loxP and FRT sites.

FIG. 9 demonstrates results of Cas9/FrtsgRNA digestion of the plasmid pLACAGRFP/tetonAqua with the approximate size of the resulting DNA fragments indicated

FIG. 10 demonstrates the alignment of the FrtsgRNA and the FRT target sequence with a PAM sequence (underlined).

FIG. 11 is a table that demonstrates the predicted fragments produced when plasmid pLACAGRFP/tetonAqua is digested by Cas9/loxPsgRNA*.

FIG. 12 demonstrates PAM-independent CRISPR cleavage in a reaction supplemented with Tth RecA, Helicase, ET SSB and T5 exonuclease.

FIG. 13 shows an alignment of the loxPsgRNA and the loxP target sequence lacking a PAM sequence (underlined).

FIGS. 14A and 14B show gel electrophoretic results demonstrating a role of Tth Rec A in PAM-independent CRISPR cleavage (FIG. 14A). pLACAGRFP/tetonAqua plasmid was digested with or without Tth Rec A (about 0.5 μg) and about 1 μL of about 20 mM ATP in a 30 μL V_{Total} reaction overnight. No differences were observed between Tth RecA and RecA on the PAM-independent CRISPR cleavage. Estimated fragment sizes are indicated with arrows in FIGS. 14A and 14B.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, microbiology, nanotechnology, organic chemistry, biochemistry, botany and the like, which are within the skill of the art. Such techniques are explained fully in the literature.

### Definitions

As used herein, “about,” “approximately,” and the like, when used in connection with a numerical variable, generally refers to the value of the variable and to all values of the variable that are within the experimental error (e.g., within the 95% confidence interval for the mean) or within +−0.10% of the indicated value, whichever is greater.

As used herein, “control” is an alternative subject or sample used in an experiment for comparison purposes and included to minimize or distinguish the effect of variables other than an independent variable.

As used herein, “diluted” used in reference to a an amount of a molecule, compound, or composition including but not limited to, a chemical compound, polynucleotide, peptide, polypeptide, protein, antibody, or fragments thereof, that indicates that the sample is distinguishable from its naturally occurring counterpart in that the concentration or number of molecules per volume is less than that of its naturally occurring counterpart.

As used herein, “separated” refers to the state of being physically divided from the original source or population such that the separated compound, agent, particle, chemical compound, or molecule can no longer be considered part of the original source or population.

As used herein, “concentrated” refers to a molecule, including but not limited to a polynucleotide, peptide, polypeptide, protein, antibody, or fragments thereof, that is distinguishable from its naturally occurring counterpart in that the concentration or number of molecules per volume is greater than that of its naturally occurring counterpart.

As used herein, “synthetic” refers to a compound that is made by a chemical or biological synthesis process that occurs outside of and independent from the natural organism from which the compound can naturally be found.

As used herein, “cDNA” refers to a DNA sequence that is complementary to a RNA transcript in a cell. It is a man-made molecule. Typically, cDNA is made in vitro by an enzyme called reverse-transcriptase using RNA transcripts as templates.

As used herein, “purified” is used in reference to a nucleic acid sequence, peptide, or polypeptide that has increased purity relative to the natural environment.

As used herein “cRNA” refers to a RNA molecule that is complementary to a DNA template and made in vitro. It is a man-made molecule.

As used herein, “electroporation” is a transformation method in which a high concentration of plasmid DNA (containing exogenous DNA) is added to a suspension of host cell protoplasts, and the mixture shocked with an electrical field of about 200 to 600 V/cm.

As used herein, “selectable marker” refers to a gene whose expression allows one to identify cells that have been transformed or transfected with a vector containing the marker gene. For instance, a recombinant nucleic acid may include a selectable marker operatively linked to a gene or insert of interest and a promoter, such that expression of the selectable marker indicates the successful transformation of the cell with the gene or insert of interest.

As used herein, “operatively linked” indicates that the regulatory sequences useful for expression of the coding sequences of a nucleic acid are placed in the nucleic acid molecule in the appropriate positions relative to the coding sequence so as to effect expression of the coding sequence. This same definition can also be applied to the arrangement of coding sequences, other functional non-coding sequences, and transcription control elements (e.g. promoters, enhancers, and termination elements), and/or selectable markers in an expression vector or other polynucleotide. This same definition can also be applied to the arrangement of individual sequences with respect to one another, where each individual sequence has a function or purpose individually and within a particular arrangement or grouping of other elements or sequences within the arrangement. “Operatively linked” does not specify a particular order of elements or sequences that may be “operatively linked” together. “Operatively linked” does not imply that any given element or sequence within the arrangement is directly next to (adjacent) or directly attached to any other particular sequence or element, although this can occur.

As used herein, “promoter” includes all sequences capable of driving transcription of a coding sequence. In particular, the term “promoter” as used herein refers to a DNA sequence generally described as the 5′ regulator region of a gene, located proximal to the start codon. The transcription of an adjacent coding sequence(s) is initiated at the promoter region. The term “promoter” also includes fragments of a promoter that are functional in initiating transcription of the gene.

As used herein, the term “vector” or is used in reference to a vehicle used to introduce an exogenous nucleic acid sequence into a cell. A vector may include a DNA molecule, linear or circular (e.g. plasmids), which includes a segment encoding a polypeptide of interest operatively linked to additional segments that provide for its transcription and translation upon introduction into a host cell or host cell organelles. Such additional segments may include promoter and terminator sequences, and may also include one or more origins of replication, one or more selectable markers, an enhancer, a polyadenylation signal, etc. Expression vectors are generally derived from yeast or bacterial genomic or plasmid DNA, or viral DNA, or may contain elements of both.

As used herein, “bind”, “binding”, and the like refer to the interaction between a paired species such as, but not limited to, enzyme/substrate, receptor/agonist or antagonist, antibody/antigen, lectin/carbohydrate, oligo DNA primers/DNA, enzyme or protein/DNA, and/or RNA molecule to other nucleic acid (DNA or RNA) or amino acid, which may be mediated by covalent or non-covalent interactions or a combination of covalent and non-covalent interactions. When the interaction of the two species produces a non-covalently bound complex, the binding that occurs is typically electrostatic, hydrogen-bonding, or the result of lipophilic interactions.

As used herein, “specific binding” refers to binding that is characterized by the binding of one member of a pair to a particular species and to substantially no other species within the family of compounds to which the corresponding member of the binding member belongs.

As used herein, “plasmid” refers to a non-chromosomal double-stranded DNA sequence including an intact “replicon” such that the plasmid is replicated in a host cell.

As used herein, “expression” describes the process undergone by a structural gene to produce a polypeptide. It is a combination of transcription and translation. Expression refers to the “expression” of a nucleic acid to produce a RNA molecule, but it is refers to “expression” of a polypeptide, indicating that the polypeptide is being produced via expression of the corresponding nucleic acid.

As used herein, “adjacent” refers to the relationship between two elements or molecules, where the two elements or molecules share a common endpoint or border.

As used herein, “identity,” is a relationship between two or more polypeptide sequences, as determined by comparing the sequences. In the art, “identity” also refers to the degree of sequence relatedness between polypeptide as determined by the match between strings of such sequences. “Identity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48: 443-453,) algorithm (e.g., NBLAST, and XBLAST). The default parameters are used to determine the identity for the polypeptides of the present disclosure.

As used herein, “polypeptides” or “proteins” are as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein “peptide” refers to chains of at least 2 amino acids that are short, relative to a protein or polypeptide.

As used herein, “transformation” or “transformed” refers to the introduction of a nucleic acid (e.g., DNA or RNA) into cells in such a way as to allow expression of the coding portions of the introduced nucleic acid.

As used herein a “transformed cell” is a cell transformed with a nucleic acid sequence.

As used herein, the term “exogenous DNA” or “exogenous nucleic acid sequence” or “exogenous polynucleotide” refers to a nucleic acid sequence that was introduced into a cell, organism, or organelle via transfection. Exogenous nucleic acids originate from an external source, for instance, the exogenous nucleic acid may be from another cell or organism and/or it may be synthetic and/or recombinant. While an exogenous nucleic acid sometimes originates from a different organism or species, it may also originate from the same species (e.g., an extra copy or recombinant form of a nucleic acid that is introduced into a cell or organism in addition to or as a replacement for the naturally occurring nucleic acid). Typically, the introduced exogenous sequence is a recombinant sequence.

As used herein, “nucleic acid sequence” and “oligonucleotide” also encompasses a nucleic acid and polynucleotide as defined above.

As used herein, “deoxyribonucleic acid (DNA)” and “ribonucleic acid (RNA)” generally refer to any polyribonucleotide or polydeoxribonucleotide, which may be unmodified RNA or DNA or modified RNA or DNA. RNA may be in the form of a tRNA (transfer RNA), snRNA (small nuclear RNA), rRNA (ribosomal RNA), mRNA (messenger RNA), anti-sense RNA, RNAi (RNA interference construct), siRNA (short interfering RNA), or ribozymes.

As used herein, “nucleic acid” and “polynucleotide” generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide includes DNAs or RNAs as described above that contain one or more modified bases. Thus, DNAs or RNAs comprising unusual bases, such as inosine, or modified bases, such as tritylated bases, to name just two examples, are polynucleotides as the term is used herein. “Polynucleotide” and “nucleic acids” also includes PNAs (peptide nucleic acids), phosphorothioates, and other variants of the phosphate backbone of native nucleic acids. Natural nucleic acids have a phosphate backbone, artificial nucleic acids may contain other types of backbones, but contain the same bases. Thus, DNAs or RNAs with backbones modified for stability or for other reasons are “nucleic acids” or “polynucleotide” as that term is intended herein.

As used herein, “nucleic acid sequence” and “oligonucleotide” also encompasses a nucleic acid and polynucleotide as defined above.

As used herein, “wild-type” is the average form of an organism, variety, strain, gene, protein, or characteristic as it occurs in a given population in nature, as distinguished from mutant forms that may result from selective breeding, recombinant engineering, and/or transformation with a transgene.

The terms “guide polynucleotide,” “guide sequence,” or “guide RNA” can refer to any polynucleotide sequence having sufficient complementarity with a target polynucleotide sequence to hybridize with the target sequence and direct sequence-specific binding of a CRISPR complex to the target sequence. The degree of complementarity between a guide polynucleotide and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). A guide polynucleotide (also referred to herein as a guide sequence and includes single guide sequences (sgRNA)) can be about or more than about 5, 10, 11, 12, 13, 14, 15, 16, 17, 18, 19, 20, 21, 22, 23, 24, 25, 26, 27, 28, 29, 30, 35, 40, 45, 50, 75, 90, 100, 110, 112, 115, 120, 130, 140, or more nucleotides in length. The guide polynucleotide can include a nucleotide sequence that is complementary to a target DNA sequence. This portion of the guide sequence can be referred to as the complementary region of the guide RNA. In some contexts, the two are distinguished from one another by calling one the complementary region or target region and the rest of the polynucleotide the guide sequence or tracrRNA. The guide sequence can also include one or more miRNA target sequences coupled to the 3′ end of the guide sequence. The guide sequence can include one or more MS2 RNA aptamers incorporated within the portion of the guide strand that is not the complementary portion. As used herein the term guide sequence can include any specially modified guide sequences, including but not limited to those configured for use in synergistic activation mediator (SAM) implemented CRISPR (Nature 517, 583-588 (29 Jan. 2015). A guide polynucleotide can be less than about 150, 125, 75, 50, 45, 40, 35, 30, 25, 20, 15, 12, or fewer nucleotides in length. It will be appreciated that the gRNA can include a crRNA portion and a trans-activating crRNA (tracrRNA).

The ability of a guide polynucleotide to direct sequence-specific binding of a CRISPR complex to a target sequence may be assessed by any suitable assay. For example, the components of a CRISPR system sufficient to form a CRISPR complex, including the guide polynucleotide to be tested, may be provided to a host cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of a CRISPR complex, including the guide polynucleotide to be tested and a control guide polynucleotide different from the test guide polynucleotide, and comparing binding or rate of cleavage at the target sequence between the test and control guide polynucleotide reactions. Other assays are possible, and will occur to those skilled in the art.

A gRNA (also called CRISPR-related RNA, crRNA) can be configured to target any DNA region of interest. The complementary region of the gRNA and the gRNA can be designed using a suitable gRNA design tool. Suitable tools are known in the art and are available to the skilled artisan. Some such tools are discussed elsewhere herein. As such, the constructs described herein are enabled for any desired target DNA so long as it is CRISPR compatible according to the known requirements for CRISPR activation. A guide polynucleotide can be selected to reduce the degree of secondary structure within the guide polynucleotide. Secondary structure may be determined by any suitable polynucleotide folding algorithm. Some programs are based on calculating the minimal Gibbs free energy. An example of one such algorithm is mFold, as described by Zuker & Stiegler ((1981) Nucleic Acids Res. 9, 133-148). Another example folding algorithm is the online webserver RNAfold, developed at Institute for Theoretical Chemistry at the University of Vienna, using the centroid structure prediction algorithm (see e.g. Gruber et al., (2008) Cell 106: 23-24; and Carr & Church (2009) Nature Biotechnol. 27: 1151-1162).

The terms “Cas9” and “Cas9 polypeptide” are used interchangeably herein to refer to an enzyme (wild-type or recombinant) that exhibits at least endonuclease activity (e.g. cleaving the phosphodiester bond within a polynucleotide) guided by a CRISPR RNA (crRNA) bearing complementary sequence to a target polynucleotide. Cas9 polypeptides are known in the art, and include Cas9 polypeptides from any of a variety of biological sources, including, e.g., prokaryotic sources such as bacteria and archaea. Bacterial Cas9 includes, Actinobacteria (e.g., Actinomyces naeslundii) Cas9, Aquificae Cas9, Bacteroidetes Cas 9, Chlamydiae Cas9, Chloroflexi Cas9, Cyanobacteria Cas9, Elusimicrobia Cas9, Fibrobacteres Cas9, Firmicutes Cas9 (e.g., Streptococcus pyogenes Cas9, Streptococcus thermophilus Cas9, Listeria innocua Cas9, Streptococcus agalactiae Cas9, Streptococcus mutans Cas9, and Enterococcus faecium Cas9), Fusobacteria Cas9, Proteobacteria (e.g., Neisseria meningitides, Campylobacter jejuni and lari) Cas9, Spirochaetes (e.g., Treponema denticola) Cas9, and the like. Archaea Cas 9 includes Euryarchaeota Cas9 (e.g., Methanococcus maripaludis Cas9) and the like. A variety of Cas9 and related polypeptides are known, and are reviewed in, e.g., Makarova et al. (2011) Nature Reviews Microbiology 9:467-477, Makarova et al. (2011) Biology Direct 6:38, Haft et al. (2005) PLOS Computational Biology I:e60 and Chylinski et al. (2013) RNA Biology 10:726-737. Other Cas9 polypeptides can be Francisella tularensis subsp. novicida Cas9, Pasteurella multocida Cas9, mycoplasma gallisepticum str. F Cas9, Nitratifractor salsuginis str DSM 16511 Cas9, Parvibaculum lavamentivorans Cas9, Roseburia intestinalis Cas9, Neisseria cinera Cas9, Gluconacetobacter diazotrophicus Cas9, Azospirillum B510 Cas9, Spaerochaeta globus str. Buddy cas9, Flavobacterium columnare Cas9, Fluviicola taffensis Cas9, Bacteroides coprophilus Cas9, mycoplasma mobile Cas9, lactobacillus farciminis Cas9, Streptococcus pasteurianus Cas9, Lactobacillus johnsonii Cas9, Staphylococcus pseudintermedius Cas9, filifactor alocis Cas9, Treponema denticola Cas9, Legionella pneumophila str. Paris Cas9, Sutterella wadsworthensis Cas9, and Corynebacter diptheriae Cas9. The term “Cas9” includes a Cas9 polypeptide of any Cas9 family, including any isoform of Cas9. Amino acid sequences of various Cas9 homologs, orthologs, and variants beyond those specifically stated or provided herein are known in the art and are publicly available, within the purview of those skill in the art, and thus within the spirit and scope of this disclosure.

### DISCUSSION

Cloning is an essential tool for genetic engineering and is the subject of intensive investigation. Many cloning techniques have been developed and most rely on cleaving DNA by using restriction enzymes. Commonly used restriction enzymes have six or eight bp recognition sequences, which have an occurrence frequency of one in every 4096 or 65536 bp in a random sequence. When used in cloning, they have two limitations. First, restriction enzymes cannot cleave at any location in a sequence that an investigator wishes. A difference of one base pair can cause major biological differences, therefore seamless cloning is desirable. Second restriction enzymes may have multiple cleavage sites, especially in a large vector of any size. In one non-limiting example, the large vector can be from 2 kb of a smaller mini vector to 2 Mb of a yeast artificial chromosomes (YACs). In most cases, unique sites are required for cloning.

To surpass the limitations with restriction enzymes, Gateway cloning, Sequence and Ligation-Independent Cloning (SLIC), Quick and Clean Cloning (QC), and Gibson assembly techniques were devised, which do not require any enzyme digestion for cloning. However, these techniques are not without their limitations, especially for large vectors. Gateway cloning does not need linear vector, but requires sequence restriction for site-specific recombination. SLIC, QC, and Gibson assembly still require restriction enzyme digestion or inverse PCR to linearize the cloning vector and do not rely on any specific vector or sequence specificity at all. The aforementioned methods only require linearized vector and linear insert(s) with homologous arms at each ends. Moreover, the use of inverse PCR to linearize the cloning vector is limited by the difficulty in amplifying vectors with high a GC content or those having repeat and long sequences, which are refractory to PCR amplification. In addition, mutations can be introduced by PCR, significantly impeding studies that depend on cloning.

At present, when large vectors are utilized in cloning, such as cosmid, baculoviral, adenoviral vectors and bacterial artificial chromosome (BAC) plasmids, direct and seamless cloning is impossible with traditional cloning methods. For example, to clone a fragment into a baculoviral and adenoviral vectors, a smaller shuttle vector is usually used to clone a fragment, and then the cloned insert is transferred into a larger vector through homologous recombination in the cells. This process is time-consuming and it takes about a month or more to obtain a correct clone.

Alternatively, these large vectors have to be engineered to have specific sequences such as attR1 and attR2 sites for Gateway cloning. Although the Gateway based cloning methods can take less than two weeks to obtain correct clones, the major disadvantage of such methods is that they can only be applied to specific vectors and require the construction of specific vectors. Moreover, it is difficult, if not impossible, to modify an existing construct by these techniques. Modifying existing constructs to suit a given study is as desirable as it is cost-effective.

With that said, described herein are methods of cloning that can utilize a clustered regularly interspaced short palindromic repeats (CRISPR) technique combined with another cloning technique, such as Gibson assembly, to clone DNA fragments seamlessly into a large vector. In some embodiments, the method can begin with synthesis of single guide RNAs (sgRNA). After the sgRNA has been generated it can be used along with Cas9 to mediate in vitro cleavage of a substrate vector. In some embodiments, a suitable single stranded DNA binding protein can be used along with Cas9 to mediate in vitro cleavage. After Cas9/sgRNA mediated vector cleavage, a DNA fragment can be seamlessly inserted into the vector via a DNA fragment (or insert) insertion technique, such as Gibson assembly. In some embodiments, the methods described herein can provide an efficient and seamless way to clone DNA into large vectors dependent or independent of PAM sequences.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

CRISPR is an adaptive immune system of bacteria to destroy naturally occurring and engineered phages and plasmid. The CRISPR-associated protein-9 (Cas9) is an endonuclease that cleaves a double-stranded DNA target site guided by a single guide RNA (sgRNA). A sgRNA is composed of a fusion of target-specific CRISPR-related sequence (crRNA) that is from the target sequence and a trans-activating CRISPR-related RNA (tracrRNA) sequence that is from the bacterial CRISPR system. A crRNA, also known as protospacer, is a sequence of usually 20-nucleotides. Current techniques require a protospacer adjacent motif (PAM) 5′-NRG (R=G or A) at the 3′ end of the crRNA sequence in the target sequence for the guided DNA recognition and cleavage by the Cas9/sgRNA complex. Thus, only about 40% of the genome can be modified using current CRISPR techniques. The CRISPR/Cas9 technique has been successfully used to edit the genomes of many species. However, it has not been demonstrated to be effective in in vitro cloning techniques, particularly for large vectors. Nor has CRISPR been demonstrated to be effective independent of a PAM sequence at the 3′ end of the crRNA sequence in the target sequence.

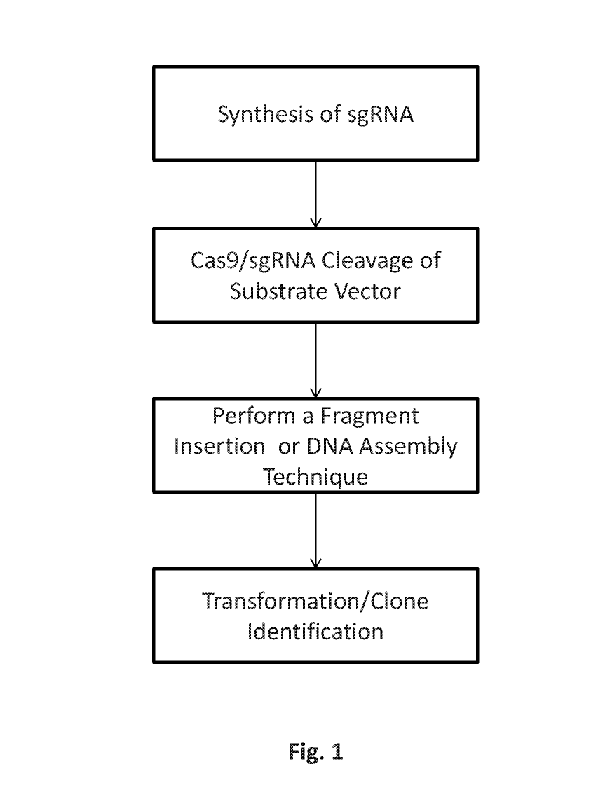

As shown in FIG. 1, one embodiment of the method can include synthesizing sgRNA, using the synthesized sgRNA along with Cas9 to mediate cleavage of a substrate vector (the vector an insert will be cloned into). Cas9/sgRNA mediated cleavage can be followed by DNA fragment insertion via a fragment insertion or DNA assembly technique. Fragment insertion and DNA assembly techniques include, but are not limited to, any or all steps or combination of steps of Gibson assembly, SLIC, sequence and ligase independent cloning (SLICE), circular polymerase extension cloning, (CPEC), simple fragment end ligation, in vitro gap-filling and nick sealing techniques, and homologous recombination techniques. The resulting vectors can be propagated and screed using standard bacterial transformation and clonal selection techniques.

Synthesizing sgRNA can include preparing a duplex DNA template for use in in vitro transcription. A duplex DNA template can be prepared via a polymerase chain reaction (PCR) reaction. The PCR reaction can include a forward primer, a reverse primer, and a template DNA.

The forward primer can contain at least three parts. The first part of the forward primer can be a RNA polymerase promoter DNA sequence that is suitable for in vitro transcription. Suitable RNA polymerase promoter sequences can include but is not limited to a T7 (5′ TAATACGACTCACTATAGG 3′) (SEQ ID NO.: 1), T3 (5′ AATTAACCCTCACTAAAGG 3′) (SEQ ID NO.: 2), or sP6 (5′ ATTTAGGTGACACTATAG 3′) (SEQ ID NO.: 3). The position in bold (+1) indicates the first nucleotide incorporated into RNA during transcription. The sequence for the RNA polymerase promoter can be operatively linked to n bases of the crRNA sequence, where n can be any number of bases from 17 bp to 21 bp in length. In some embodiments, n can be 19 bp.

The crRNA sequence is complementary to a target sequence within the substrate vector. In this way, the substrate vector can be prepared for specific, direct, and seamless cloning of an insert. Tools are publicly available online to assist in determining suitable crRNA sequences. The online tool can help identify inter alia PAM sequences and sequences adjacent to PAM sequences in the substrate vector, which facilitate Cas9 cleavage. Exemplary crRNA and sgRNA design tools are shown in Table 1. In other embodiments, the crRNA sequence/target sequence is not determined based on the sequence's proximity to a PAM sequence in the substrate vector and can be located anywhere within a substrate vector. Suitable crRNA polynucleotides can be generated by techniques generally known in the art, such as de novo DNA synthesis.

TABLE 1

| gRNA design | | |
|---|---|---|
| Tool | Reference | Comments |
| Cas-OFFinder | Jin-Soo Kim Lab, Center for | www.rgenome.net |
| | Genome Engineering, | identifies gRNA target sequences from an |
| | Department of Chemistry, | input sequence and checks for off-target |
| | Seoul National University, | binding. Currently supports: Drosophila, |
| | Seoul, Korea | Arabidopsis, zebrafish, C. elegans, |
| | | mouse, human, rat, cow, dog, pig, Thale |
| | | cress, rice (Oryza sativa), tomato, corn, |
| | | monkey (macaca mulatta). |
| Cas-Designer | Jin-Soo Kim Lab, Center for | www.rgenome.net |
| | Genome Engineering, | searches for targets that maximize |
| | Department of Chemistry, | knockout efficiency while having a a low |
| | Seoul National University, | probability of off-target effects. Cas- |
| | Seoul, Korea | Designer integrates information from the |
| | | Kim Lab's Cas-OFFinder and |
| | | Microhomology predictor. |
| CRISPR-ERA | Qi Lab, Stanford University | a sgRNA design tool for genome editing, |
| | School of Medicine, Palo Alto, | as well as gene regulation (repression and |
| | CA. | activation). Genome support for bacteria |
| | | (E. coli, B. subtilis), yeast (S. cerevisiae), |
| | | worm (C. elegans), fruit fly, zebrafish, |
| | | mouse, rat, and human. |
| CCTop | Stemmer, M., Thumberger, T., | http://crispr.cos.uni-heidelberg.de/ |
| | del Sol Keyer, M., Wittbrodt, J. | Identifies candidate sgRNA target sites by |
| | and Mateo, J. L. CCTop: an | off-target quality. Validated for gene |
| | intuitive, flexible and reliable | inactivation, NHEJ, and HDR. Reference |
| | CRISPR/Cas9 target prediction | genomes include Arabidopsis, C. elegans, |
| | tool. PLOS ONE (2015). doi: | sea squirt, cavefish, Chinese hamster, |
| | 10.1371/journal.pone.0124633 | fruit fly, human, rice fish, mouse, silk |
| | | worm, stickleback, tobacco, tomato, frog |
| | | (X. laevis and X. tropicalis), and zebrafish. |
| Off-Spotter | Pliatsika, V, and Rigoutsos, I | https://cm.jefferson.edu/Off-Spotter/ |
| | (2015) “Off-Spotter: very fast | Program for designing optimal gRNAs. |
| | and exhaustive enumeration of | Provides feedback on number of potential |
| | genomic lookalikes for | off-targets, target's genomic location, and |
| | designing CRISPR/Cas guide | genome annotation. Available genomes |
| | RNAs” Biol. Direct 10(1): 4 | are human (hg19 & hg38), mouse |
| | | (mm10), and yeast (strain w303). |
| CRISPR | Sergey Prykhozhij at the IWK | http://www.multicrispr.net/ |
| MultiTargeter | Health Centre and Dalhousie | Can be used to identify novel gRNA target |
| | University. | sites in a single gene, as well as a target |
| | | site common to a set of similar |
| | | sequences. Organisms include human, |
| | | mouse, rat, chicken, frog, zebrafish, fly, |
| | | worm, Japanese rice fish, maize, |
| | | Arabidopsis, and rice. Proof-of-concept |
| | | performed in zebrafish. |
| ZiFiT Targeter | Sander, J. D., Zaback, P. Z., | http://zifit.partners.org/ZiFiT/ |
| Version 4.2 | Joung, J. K., Voytas, D. F., | Originally developed to identify zinc finger |
| | Dobbs, D. (2007) Zinc Finger | nuclease sites, this tool has been |
| | Targeter (ZiFiT): an engineered | expanded to identify potential DNA target |
| | zinc finger/target site design | sites for TALEs and CRISPR/Cas |
| | tool. Nucleic Acids Research, | |
| | 35, W599-605 and Sander, J. D., | |
| | Maeder, M. L., Reyon, D., | |
| | Voytas, D. F., Joung, J. K., | |
| | Dobbs, D. (2010) ZiFiT (Zinc | |
| | Finger Targeter): an updated | |
| | zinc finger engineering tool. | |
| | Nucleic Acids Research, | |
| | 38: W462-468; | |
| CRISPR direct | Naito Y, Hino K, Bono H, Ui-Tei K. | http://crispr.dbcls.jp/ |
| | (2015) CRISPRdirect: | From the Database Center for Life |
| | software for designing | Science (DBCLS) in Japan; Identify |
| | CRISPR/Cas guide RNA with | candidate gRNA target sequences in an |
| | reduced off-target sites. | input sequence, which can be an |
| | Bioinformatics, 31, 1120-1123. | accession number, genomic location, |
| | | pasted nucleotide sequence, or a |
| | | sequence text file you upload. Currently |
| | | supports: Human, mouse, rat, marmoset, |
| | | pig, chicken, frog (X. tropicalis and X. laevis), |
| | | zebrafish, sea squirt, Drosophila, |
| | | C. elegans, Arabidopsis, rice, sorghum, |
| | | silkworm, and budding and fission yeast. |
| Feng Zhang lab's | Feng Zhang Lab, | http://crispr.mit.edu/ |
| Target Finder | Massachusetts Institute of | Identifies gRNA target sequences from an |
| | Technology 2015 | input sequence and checks for off-target |
| | | binding. Currently supports: Drosophila, |
| | | Arabidopsis, zebrafish, C. elegans, |
| | | mouse, human, rat, rabbit, pig, possum, |
| | | chicken, dog, mosquito, and stickleback. |
| E-CRISP | Michael Boutros Lab at the | http://www.ecrisp.org/ECRISP/designcrispr.html |
| | German Cancer Research | Identifies gRNA target sequences from an |
| | Center Heidelberg, Germany | input sequence and checks for off-target |
| | | binding. Currently supports: Drosophila, |
| | | Arabidopsis, zebrafish, C. elegans, |
| | | mouse, human, rat, yeast, frog, |
| | | Brachypodium distachyon, Oryza sativa, |
| | | Oryzias latipes |
| CasFinder: | Aach J, Mali P, Church GM. | http://arep.med.harvard.edu/CasFinder/ |
| Flexible Algorithm | 2014. CasFinder: Flexible | From the Church Lab, a program that |
| for identifying | algorithm for identifying specific | identifies gRNA target sequences from an |
| specific Cas9 | Cas9 targets in genomes. | input sequence, checks for off-target |
| targets in | bioRxiv doi: 10.1101/005074 | binding and can work for S. pyogenes, S. thermophilus |
| genomes | | or N. meningitidis Cas9 |
| | | PAMs. Currently supports: mouse and |
| | | human. |
| CRISPR Optimal | Gratz, S. J.*, Ukken, F. P.*, et al. | http://tools.flycrispr.molbio.wisc.edu/targetFinder/ |
| Target Finder | (2014) Genetics. | This software from the O'Connor-Giles |
| | | Lab identifies gRNA target sequences |
| | | from an input sequence and checks for |
| | | off-target binding. Currently supports over |
| | | 20 model and non-model invertebrate |
| | | species. |

In some embodiments, the crRNA polynucleotide can be cloned into a vector that contains a tracRNA sequence and/or other components of the sgRNA. The suitable vector can contain other segments of the forward and/or reverse primers. The crRNA polynucleotide can be operatively linked to n bases of tracrRNA, where n can be about 80 bp to about 172 bp. In some embodiments, the tracrRNA sequence in the forward primer can be about 20 bp in length. The reverse primer can be a suitable reverse primer that would result in amplification of the region of interest in the template DNA.

The duplex DNA template is generated by performing a PCR reaction. The PCR reaction can include an initial denaturing step at about 98° C. for about 2 to about 5 minutes. This can be followed by about 30 to about 5 cycles of the following: about 10 sec to about 30 sec at about 98° C., about 15 sec to about 30 sec at anywhere from about 50° C. to about 60° C., and about 1 minute to about 2 minutes at about 68° C. This can be followed with a final extension for about 1 to about 15 minutes at about 68° C. to about 72° C. In other embodiments, the PCR reaction can include an initial denaturing step at about 95° C. for about 30 sec to about 2 to about 5 minutes. This is followed by about 30 cycles of the following: about 15 sec to about 1.5 minutes at about 95° C., about 15 sec to about 2 minutes at anywhere from about 50° C. to about 68° C.; and 15 sec to about 2 minutes at about 72° C. This can be followed with a final extension for about 1 minute to about 15 minutes at about 72° C. The template for the PCR reaction can be any suitable template to generate the sgRNA as described herein. In some embodiments, the template can be the pX330 or other suitable vectors that have the crRNA sequences cloned such as, but not limited to, pX330-LAsg vector.

The duplex DNA template can then be used in an in vitro transcription reaction to generate the sgRNA, which is a cRNA molecule that contains at least the crRNA and the tracrRNA. In vitro transcription can be carried out by using methods generally known in the art. The polymerase used in the in vitro transcription reaction corresponds to the sequence of the promoter in the duplex DNA template. The cRNA produced can be further purified.

In other embodiments, the duplex DNA template for in vitro transcription can be chemically synthesized de novo. In this instance, the duplex DNA template can contain a RNA polymerase promoter sequence, such as T7, T3, or Sp6, operatively linked to about 17 bp to about 20 bp of a crRNA sequence, which can be operatively linked to a tracrRNA.

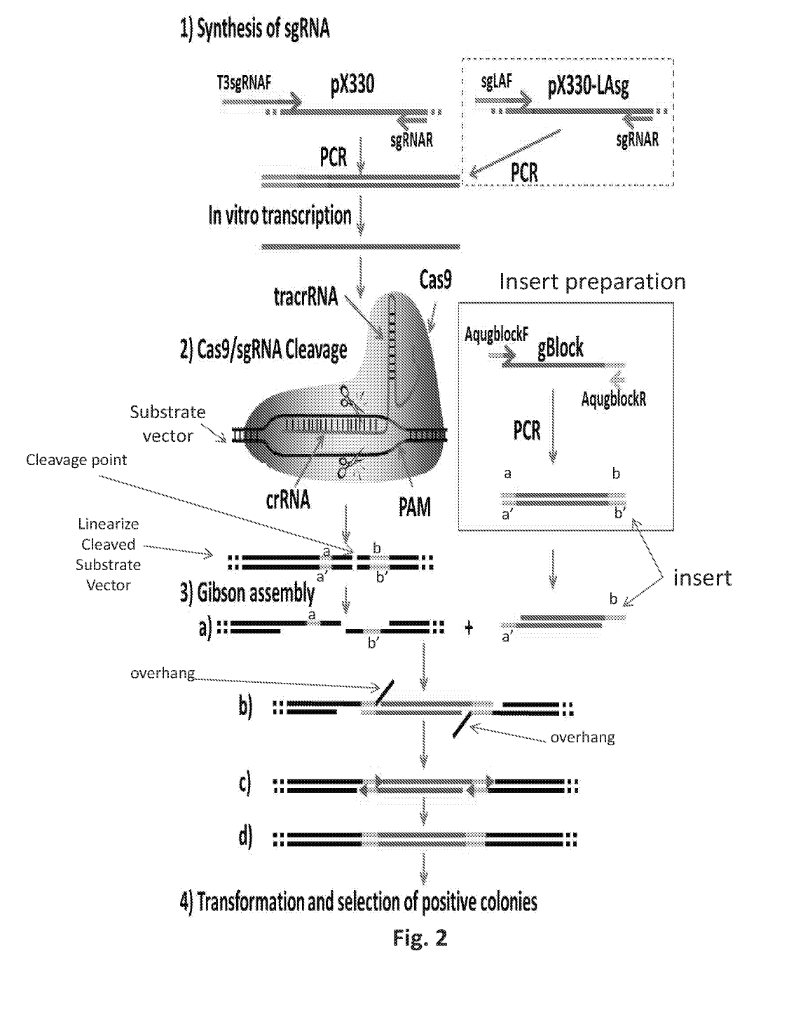

The sgRNA from in vitro transcription can be used as a template for Cas9/sgRNA mediated cleavage reaction as shown in FIG. 2. It will be appreciated that while FIG. 2 demonstrates embodiments of the methods described herein using Gibson assembly as the DNA assembly method, it will be appreciated that any fragment insertion or DNA assembly method or step(s) thereof can be used in place of Gibson assembly. Other suitable fragment insertion and DNA assembly methods are described elsewhere herein (e.g. in relation to FIG. 1). The cRNA, can be mixed with Cas9 nuclease. The final concentration of Cas9 nuclease can range from about 1 nM to about 50 nM in the Cas9/sgRNA mediated cleavage reaction. In some embodiments, the final concentration of Cas9 nuclease in the Cas9/sgRNA mediated cleavage reaction is about 30 nM. The total volume of the Cas9/sgRNA mediated cleavage reaction can range from about 10 μL to about 100 μL. The Cas9/sgRNA mediated cleavage reaction can also contain an amount of sgRNA. The absolute amount of sgRNA included in the reaction will vary such that the sgRNA is included in the Cas9/sgRNA mediated cleavage reaction at a final concentration between about 3 nM and about 300 nM. Additionally, the Cas9/sgRNA mediated cleavage reaction mixture can optionally contain a suitable reaction buffer. The Cas9/sgRNA mediated cleavage reaction also can also contain an amount of the substrate vector. The final concentration of the substrate vector in the reaction can range from about 1 nM to about 30 nM. In some embodiments, the final concentration of substrate vector in the reaction can be about 30 nM. In further embodiments, the Cas9/sgRNA mediated cleavage reaction contains an amount of a suitable single stranded DNA binding protein. Suitable single stranded DNA binding proteins are those proteins that have single stranded DNA binding properties and facilitate PAM-independent Cas9 cleavage. Such single strand DNA binding proteins include, but are not limited to, Tth RecA, Helicase, Extreme Thermostable single stranded DNA Binding protein, Escherichia coli (E. coli) single stranded DNA Binding protein and Rec A, and RecA. The amount of the suitable single stranded binding protein can range from about 0.1 μg to about 10 μg. other embodiments, the Cas9/sgRNA mediated cleavage reaction also includes an amount of ATP. The amount of ATP can be such that the final concentration of ATP in the Cas9/sgRNA mediated cleavage reaction ranges from about 0 mM to about 50 mM.

The Cas9/sgRNA cleavage reaction can be pre-incubated at about 37° C. for about 0 to about 30 min. In some embodiments, the Cas9/sgRNA cleavage reaction is pre-incubated for about 10 minutes. After the optional pre-incubation, the substrate vector was digested in the Cas9/sgRNA cleavage reaction by incubating the Cas9/sgRNA cleavage reaction for about 1 to about 72 hours. In some embodiments, the suitable single stranded DNA binding protein is added to the Cas9/sgRNA cleavage reaction after the optional pre-incubation. The Cas9/sgRNA cleavage reaction can produce a linearized cleaved substrate vector having a cleavage point as shown in FIG. 2. The Cas9 endonuclease can cleave the substrate vector at a position within the substrate vector that is complementary to the sgRNA sequence and adjacent to a PAM sequence within the substrate vector as shown in FIG. 2. The point at which Cas9 cleaves the substrate vector is referred to herein as the cleavage point. The linearized cleaved substrate vector can be separated and obtained via gel electrophoresis and subsequent purification from the gel.

A polynucleotide insert to be cloned into the substrate vector can be prepared using PCR amplification or other suitable technique, which will be appreciated by those of skill in the art. The forward and/or reverse primers used to amplify the desired insert sequence can incorporate sequences complementary to the substrate vector such that the insert will be placed at or near the site of Cas9 cleavage in the cleaved substrate vector. This is also shown in FIG. 2. The sequence of “a” is complementary to “a primed (a′)” and the sequence of “b” is complementary to “b primed (b′).” Therefore, when the insert is subjected to Gibson assembly, the a′ will bind with the sequence of a in the linearized cleaved substrate vector and b′ of the substrate vector will bind with b of the insert. This is also shown in FIG. 2. The a/a′ and b/b′ regions can be about 20 to about 40 base pairs in length. The exact sequence of the primers used to generate the insert can be determined by one of ordinary skill in the art based at least upon the parameters described herein and others known in the art. Other methods of generating inserts with ends complementary to the substrate vector near or at the point of Cas9 cleavage are known in the art. After generation, the insert can be optionally purified by a suitable technique, including but not limited to gel electrophoresis and purification and phenol/chloroform extraction and purification.

After Cas9/sgRNA mediated cleavage of the substrate vector and preparation of the insert, Gibson assembly (Gibson cloning) can be performed using methods generally known in the art. It will be appreciated that while FIG. 2 demonstrates embodiments of the methods described herein using Gibson assembly as the DNA assembly method, it will be appreciated that any fragment insertion or DNA assembly method or step(s) thereof can be used in place of Gibson assembly. Other suitable fragment insertion and DNA assembly methods are described elsewhere herein (e.g. in relation to FIG. 1). Techniques for performing other fragment insertion and DNA assembly methods will be appreciated by those of skill in the art.

In embodiments, employing Gibson assembly an amount of the linearized cleaved substrate vector can be incubated at least with an amount of an exonuclease, an amount of a DNA polymerase, and an amount of a DNA ligase. It will be appreciated by those of skill in the art that the linearized substrate vector can be incubated with other compositions, compounds, reagents, enzymes, etc., as necessary depending on the fragment insertion technique or DNA assembly technique being employed. Those compositions, compounds, reagents, enzymes, will be appreciated by those of skill in the art.

In any embodiment, a total of about 0.02-0.2 pmols of DNA fragments can be used in the reaction. In some embodiments, 50-100 ng of vector can be incubated with an excess of insert(s) relative to the amount of vector. The insert(s) can be 2-10, 2-5 fold, 2-3 fold in excess relative to the amount of vector. In embodiments, the ratio of substrate vector to insert in the fragment insertion or DNA assembly reaction can be varied and can range from about 1:1 to about 1:10 to about 10:1, substrate vector to insert. In some embodiments, a commercially available kit for performing the fragment insertion or DNA assembly method (e.g. a Gibson cloning kit) can be utilized. The fragment insertion or DNA assembly reaction can be carried out at about 25° C. to about 50° C. for about 15 minutes to about 16 hours. This can produce a substrate vector containing an insert at or near the cleavage site. While the Gibson assembly process is demonstrated in FIG. 2, it will be appreciated that the method can utilize other fragment insertion or DNA assembly techniques as described elsewhere herein. In embodiments employing Gibson assembly, overhanging edges can be removed, any gaps can be filled in, and the insert can be ligated into the linearized substrate vector.

After fragment insertion, a suitable competent cell can be transformed using a suitable transformation technique, which will depend inter alia on the cell line used. Suitable competent cells include DH5α™, SoluBL21™ E. coli, CloneCatcher™ Gold DHSG E. coli, TurboCells™ E. coli, and TOP10. Suitable transformation techniques are generally known in the art. After transformation, cells are grown and cells that contain the plasmid carrying the insert are selected. Positive clones can be identified using a suitable marker such as antibiotic resistance or X-gal sensitivity, or via PCR screening, restriction enzyme digest, and/or sequencing. Positive clones can be grown and plasmid DNA can be obtained using a standard plasmid DNA preparation and purification method generally known in the art.

It will be understood by one of skill in the art that the methods described herein can be applied to existing vectors to allow them to be modified without reliance upon restriction enzymes. In other words, the methods can be applied to substrate vector that is a vector already in existence that may or may not have been previously modified by cloning. Further it will be instantly appreciated that some of the methods or steps thereof can be applied to modify a genome or other sized vectors. For example, some of the methods or steps thereof for PAM-independent Cas9 mediated DNA cleavage can be utilized to specifically cleave a genome (substrate genomic DNA) or other sized vectors.

### Examples

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1: CRISPR/Gibson Cloning into a 22 kb Target Vector

Materials and Methods

The general strategy includes four steps, which are outlined in FIG. 1. A specific strategy that was used clone into a large vector of about 22 kb is outlined in FIG. 2.

sgRNA Synthesis

First, sgRNA was synthesized. Reagents and oligo synthesis were as follows: All enzymes and other reagents were purchased from New England Biolabs (NEB, Ipswich, Mass., USA) unless otherwise specified. All DNA oligos were synthesized by Integrated DNA Technologies (Coralville, Iowa, USA) or Eurofins Genomics (Huntsville, Ala., USA). All primer sequences can be found in FIG. 7. For synthesizing a specific sgRNA that targets T3 promoter without cloning, a forward primer (T3gRNAF) contained three parts: a T7 promoter sequence, CRISPR-RNA (crRNA) sequence, and the first 20 bases of the tracrRNA sequence from the pX330 empty vector (Addgene plasmid 42230) (FIG. 7). To amplify a sgRNA from a pX330-derived vector that contains the desired sgRNA, the forward primer (sgLAF) contained only the first two parts stated above. The same reverse primer (sgRNAR) was used in both cases.

For sgRNA cloning, a guide sequence was designed using the online CRISPR design tool available through the Massachusetts Institute of Technology. Two oligos (mLAsgF and mLAsgR) were synthesized and annealed to form a double strand fragment with the desired overhangs (sequences in lower case in FIG. 7). for cloning into the pX330 vector. The two oligos were diluted and mixed together at final concentration of 10 μM and denatured at 95° C. for 5 min in a PCR machine. The machine was then turned off and the temperature of the tube was cooled to room temperature over 30 minutes. The crRNA sequence was cloned into the pX330 vector using the following protocol: μg of pX330 was digested with 10 units of Bbsl (Thermo Scientific, Waltham, Mass., USA) with 2 μl of 10× Buffer G in the presence of 400 units of T4 ligase, 1 μl of annealed oligo (10 μM stock), and 1 mM of ATP at 37° C. overnight. Next, 2 μl of the ligation reaction was used to transform competent cells. Positive clones were selected by Bbsl and Scal digestion.

Next the T7 DNA template was amplified via PCR. PCR was used to amplify the DNA template for in vitro synthesis of the sgRNA by in vitro transcription with T7 RNA polymerase. The PCR reaction mixture contained 2 μl of 10×Pfx50™ PCR Mix, 2.4 μl of 2.5 mM dNTP Mix, 1.2 μl of 10 μM forward and reverse primer (T3gRNAF and sgRNAR, FIG. 7) mix, 0.4 μl of plasmid template (pX330, 2.2 ng/μl), and 0.4 μl of Pfx50™ DNA Polymerase (5 U/μl) (Life Technologies, Grand Island, N.Y., USA). Sterile distilled water was added to bring the total reaction volume to 20 μl. The PCR cycling parameters were 94° C. 2 min, 5 cycles of 94° C. 15 s and 68° C. 20 s, 5 cycles of 94° C. 15 s and 66° C. 10 s, 68° C. 20 s and 25 cycles of 94° C. 15 s, 63° C. 10 s and 68° C. 20 s, and one cycle of 68° C. 10 min. When amplifying the sgRNA from the plasmid cloned above, primers sgLAF and sgRNAR were used. PCR products were extracted with phenol/chloroform and then purified by an S-300 microSpin column (GE Healthcare Bio-Sciences, Pittsburgh, Pa., USA) following the manufacturer's instructions.

Finally the sgRNA was transcribed in vitro. The in vitro transcription was conducted for 4 hr at 37° C. in a 50 μl reaction mixture containing 5 μl of 10×RNAPol reaction buffer, 2.5 μl of 10 mM NTP Mix, 0.5 μl of 10 mg/ml BSA, 1 μl of murine Rnase inhibitor (40 U/μl), 40 μl of purified PCR product (17 ng/μl), and 1 μl of T7 RNA Polymerase (50 U/μl). The template DNA did not appear to affect the Cas9 digestion, so it was not removed. The transcription product was purified as in step 1 or used directly without any obvious adverse effects.

Linearization of Cloning Vector Using sgRNA and Cas9

The digestion was carried out in a 30 μl reaction mixture composed of 3 μl of 10×Cas9 nuclease reaction buffer, 126 ng (300 nM) of sgRNA, and 1μ of 1 μM Cas9 nuclease (NEB). Sterile distilled water was added to bring the total reaction volume to 30 μl. The final concentration of Cas9 nuclease was 30 nM. There is no unit definition for the enzyme from the manufacturer (NEB). The mixture was pre-incubated for 10 min at 37° C., 30 nM substrate plasmid A1 DNA (pLACAGRFPTetOn, 21683 bp. The position of crRNA sequence is 12651-12669 was added, and the mixture was incubated for 1 hr (as recommended by the manufacturer), overnight, or 72 hrs following the manufacturer's protocol. The vector that was digested overnight was used for cloning. The Cas9 digested vector was purified as in step 1.

Gibson Cloning

The insert was PCR amplified in a 20 μl reaction mixture composed of 4 μl of 5× PrimeSTAR GXL buffer, 1.6 μl of 2.5 mM dNTP mix, 0.4 μl of the 10 μM forward and reverse primers, 0.8 μl of plasmid template (pX330, 2.2 ng/μl), and 0.4 μl of PrimeSTAR GXL DNA polymerase (5 U/μl) (Clontech Laboratories, Mountain View, Calif., USA). Sterile distilled water was added to bring the total reaction volume to 20 μl. The PCR cycling parameters were 94° C. 2 min, 5 cycles of 94° C. 15 s and 72° C. 20 s; 5 cycles of 94° C. 15 s and 70° C. 20 s, 26 cycles of 94° C. 15 s and 68° C. 20 s, and one cycle of 68° C. 10 min, using the primers AqugblockF and AqugblockR. The PCR product of the insert (783 bp) and the Cas9/sgRNA digested vector were phenol/chloroform extracted and purified by an S-300 microspin column as performed above. The purified vector (63 ng) and insert (47 ng) mixture (10 μl) was mixed with 10 μl of Gibson Assembly® Master Mix and incubated at 37° C. for 1 hr. The quick and clean cloning (QC) method also was used as described previously. See Thieme, F., et al., Quick and clean cloning: a ligation-independent cloning strategy for selective cloning of specific PCR products from non-specific mixes. PLoS One, 2011. 6(6): p. e20556.

Bacterial Transformation and Selection of Positive Colonies

Two μl of Gibson or QC reaction was used to transform 100 μl of home-made DH5α competent cells following standard transformation protocol. The mini-preparation of plasmid DNAs was carried out using the Zymo-Spin™ II columns (Zymo Research Corporation, Irvine, Calif., USA). Positive clones were identified by restriction enzyme digestion and sequencing.

Results

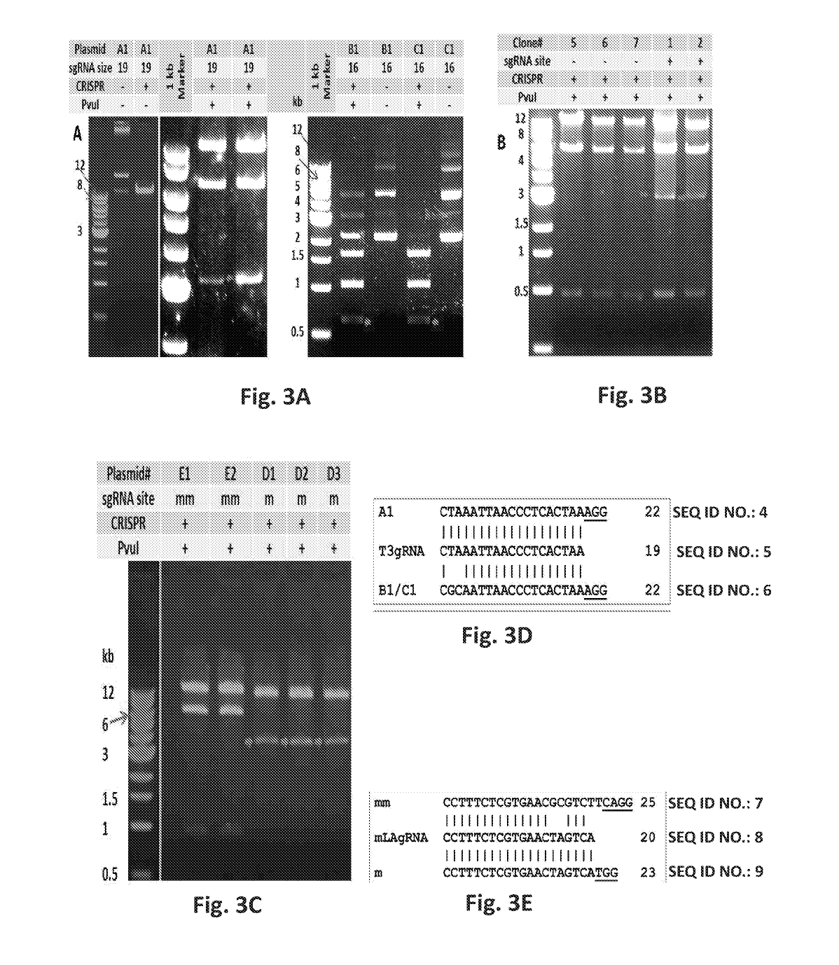

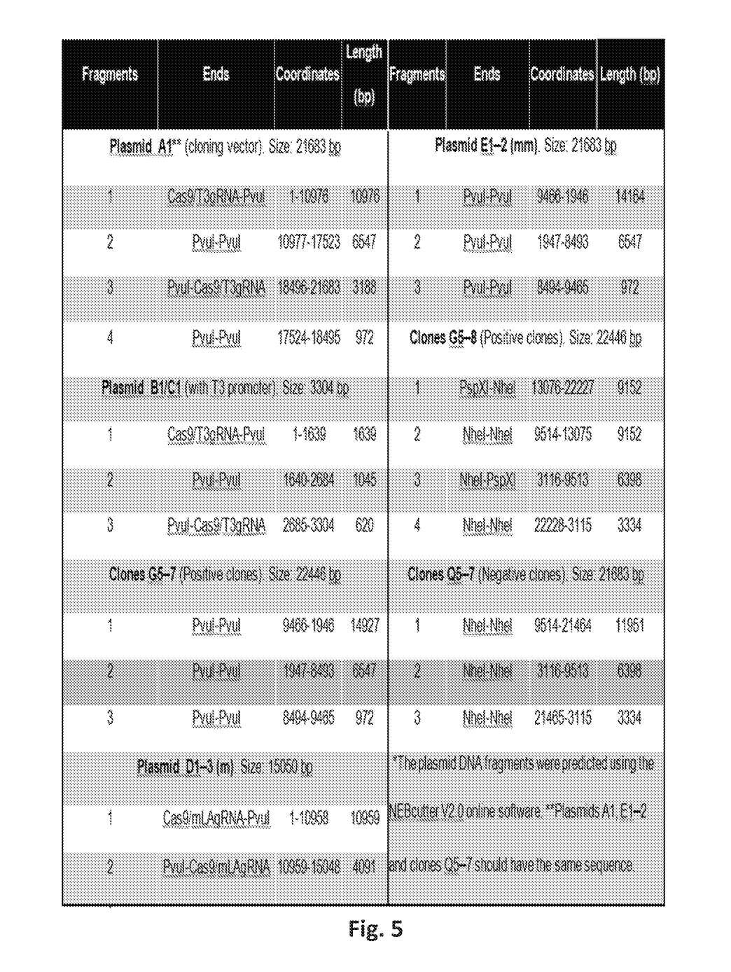

Here, three plasmids were used to determine the specificity of CRISPR cleavage. Plasmid A1 is a 22 kb target vector that has a 19 bp sequence which is fully matched with the 19 bp crRNA sequence (FIG. 3D). The crRNA sequence contains 16 bp of the T3 promoter. Plasmids B1 & C1 have the T3 promoter sequence (16 bp) which is fully matched with the 3′ 16 bp of the 19 bp crRNA sequence (FIG. 3D). The results demonstrate that the Cas9/T3gRNA can specifically digest all clones. When combined with the restriction enzyme Pvul, the correct CRISPR-digested band was present as predicted for each plasmid (FIGS. 3A and 5). The digestion for plasmid A1 was complete after about 1 hr. DNA degradation or non-specific digestion was not observed after prolonged incubation (up to about 72 hr), suggesting that the Cas9 digestion is specific and should be suitable for cloning. Digestion for plasmids B1 & C1 did not appear to be complete after about 72 hr. After about one hour of digestion recommended by the manufacturer, only a small fraction of plasmid was digested (data not shown). The sequence alignment demonstrates that B1 or C1 only had two mismatches compared to the A1 vector (FIG. 3D). This suggests, without being bound to theory, that the Cas9/sgRNA cleavage of the sequence with 16 bp match was less efficient than that of the sequence with 19 bp match, and that the mismatches distal to the PAM sequence can severely reduce the cleavage efficiency. It is possible that mismatches thermodynamically destabilize the DNA/Cas9/sgRNA complex, which was unexpected because previous reports demonstrated that there was no significant difference of cleavage efficiency between 17 base and 20 base sgRNAs in vivo. A prolonged digestion or higher concentration of Cas9/sgRNA can be used to digest all of the B1 or C1 DNA.

Furthermore, another sgRNA sequence-mediated CRISPR digestion was tested on two vectors. The second vector (“E”, in FIG. 3C) was modified from the first vector (“D”, in FIG. 3C) and had an insertion that interrupted the LA crRNA sequence. The D vector had the crRNA sequence (m, match), while the E vector did not have the crRNA sequence but a sequence that had several mismatches (mm) with the crRNA sequence (FIG. 3E). The Cas9/LAsgRNA cleavage combined with Pvul digestion show that the Cas9/sgRNA complex cleaves only the plasmid with matched sequences (FIG. 2).

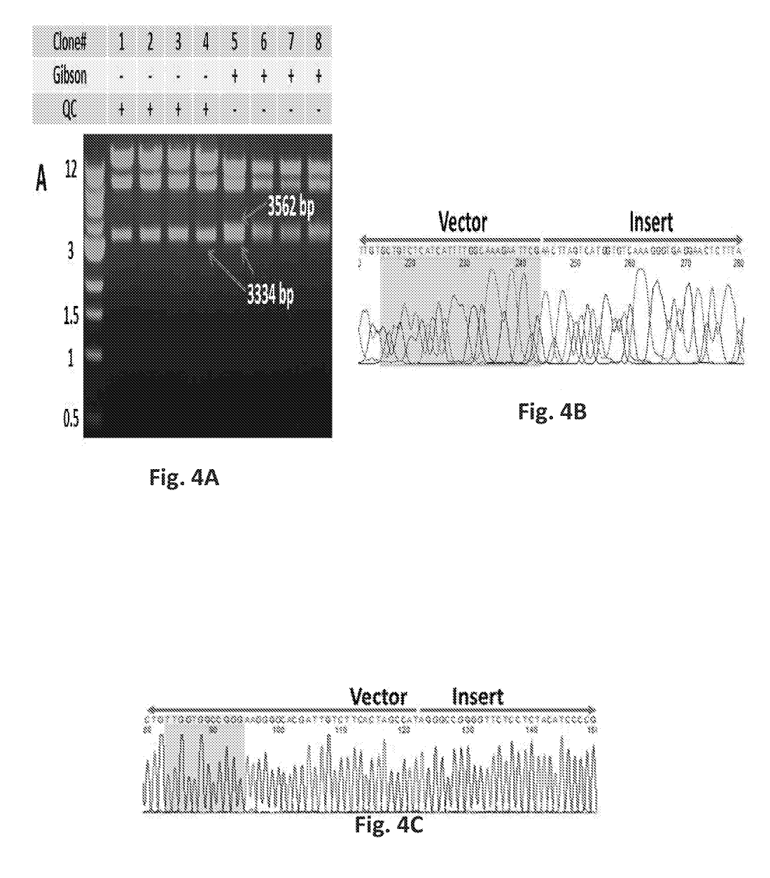

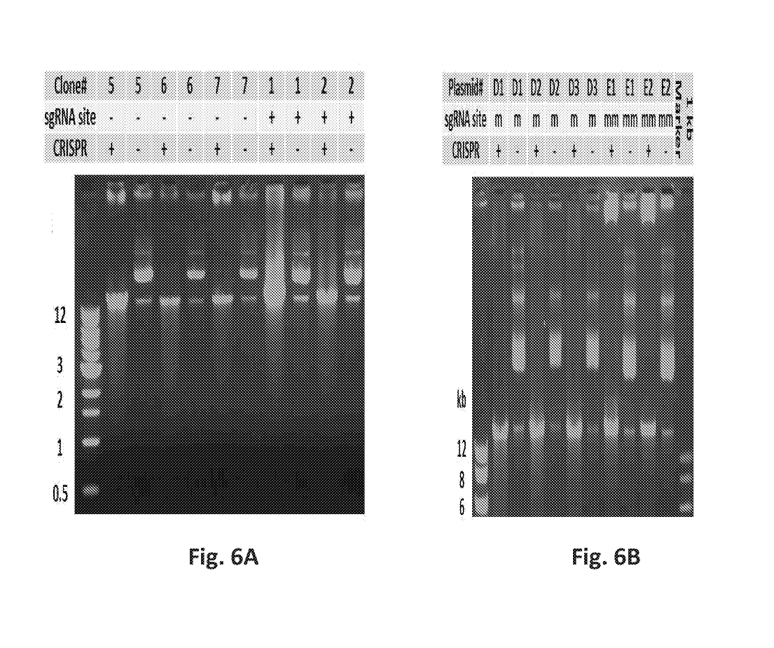

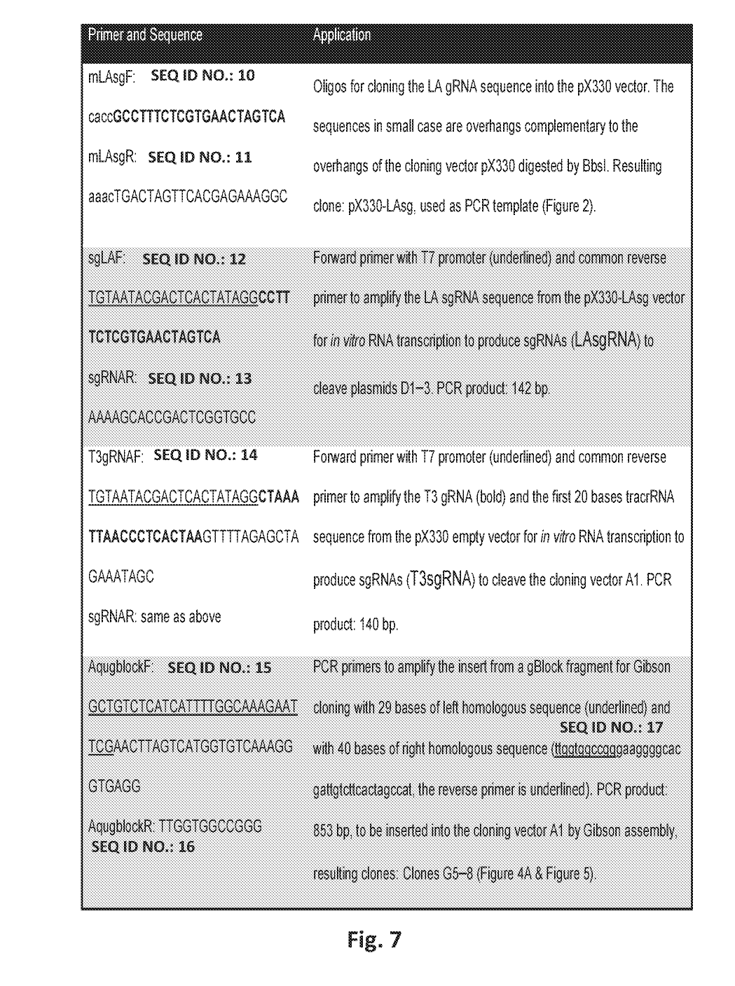

Here, the Gibson cloning produced 287 colonies, while the QC plate produced only a few colonies. Since the insert had one PspXI site, a successful cloning would introduce a unique PspXI into the vector. We used PspXI and NheI digestion to identify positive clones, which produced two unique fragments (FIG. 5). The four clones (G5-8) from Gibson cloning were all positives (pLACAGRFP/tetonAqua), while the four clones (Q1-4) from QC were all negatives, as confirmed by restriction enzyme mapping and sequencing showing bands with expected sizes (FIG. 4B) and the two junctions of the vector and insert were seamlessly joined (FIG. 4C). The Cas9/T3sgRNA cleavage combined with Pvul digestion also identified positive clones from negative clones (FIG. 3B). CRISPR-only treatment resulted in “linearization” of plasmids that do not have the sgRNA sequence, suggesting that Cas9 may have topoisomerase activity that changes plasmid conformation (FIGS. 6A and 6B). Gibson assembly typically requires an exact match of the homology region at the end of the linearized vector and linear insert. The vector linearized by the Cas9/sgRNA in FIG. 3A has 3′ overhangs after chewing by exonuclease and annealing during Gibson assembly (FIG. 2). These protruding overhangs can be removed by DNA polymerase before the DNA is ligated. The enzymes in the Gibson Assembly® Master Mix were not disclosed by the company. However, in the original Gibson assembly protocol, Phusion DNA polymerase, which has 3′ to 5′ exonuclease activity was used. The Gibson Assembly® Master Mix may contain the same enzyme or a similar one which has the activity to remove these heterologous regions before filling in any gaps between the homologous regions. Therefore, the two homologous sequences are not necessarily required to be located at the very end of the sequence produced by the Cas9/sgRNA digestion as required by the manufacturer's Gibson cloning manual. In fact, in other homologous recombination-based protocols, up to several hundred bp of heterologous sequences flanking the homologous sequence from both ends can be effectively removed. Here, the heterologous sequences at the two ends are 18 bp and 12 bp, and they did not appear to affect the Gibson cloning. This property allows one to choose homologous sequences away from the Cas9/sgRNA cleaved ends and to clone seamlessly using this method.

The specificity of the Cas9/sgRNA digestion was determined by a 19 bp crRNA sequence in a vector. A PAM (5′-NRG) sequence is typically required at the 3′ end of the crRNA, which can be any sequence. Thus, a crRNA sequence can be found close to a targeted site anywhere in the vector as the crRNA sequence can be from both strands. The NRG frequency in a random double strand DNA sequence is one in every four bp. The sgRNA guided Cas9 digestion can be highly specific as the frequency of a 16 bp crRNA sequence is one in every 4.3 billion bp. This is larger than the 3.3 billion bp human genome. Although off-target sequences may have up to five mismatches, using a shorter crRNA sequence can improve specificity but may be less efficient as the 16 bp used in this study. Consistent with these results, a minimum of 17 nucleotides of complementarity was useful for efficient RNA-guided nucleases (RGN) activity. No difference in digestion efficiency between the 19 bp crRNA and the 20 bp crRNA used here was observed. A sequence (RCGGH [R=A or G, H=T, A, C]) was found to favor Cas9 cleavage over the canonical NGG sequence. The PAM-proximal sequence appeared to be relevant as demonstrated by the findings that Cas9/gRNA complex first binds to the PAM and then unwinds the DNA adjacent to the PAM. The two corresponding five bp sequences in this study were AAGGG for T3 crRNA and ATGGC for mLA crRNA. No digestion problems were observed with these sequences.

In sum, demonstrated herein is the successful cloning a fragment into a large vector at high efficiency by using the CRISPR/Cas9 nuclease combined with Gibson assembly. The results demonstrated that CRISPR/Cas9 technique can be used as “a restriction enzyme” to cleave DNA in vitro for cloning, without being burdened by the limitations of a restriction enzyme. Moreover, this method does not require the generation specific vectors, or to linearize a vector, which lacks any desired restriction enzyme site, by inverse PCR. Further the method is less time consuming and more convenient than traditional cloning methods. The whole process can be completed within about one week. Several reagents, including Cas9 are commercially available. Oligos can be synthesized in about one day, sgRNA can be synthesized on another day, and the Cas9/sgRNA digestion can be carried out overnight. Gibson cloning and transformation can be finished on the third day. Colony pickup, culturing, the mini-preparation of DNA, and identification can be done on the fourth and fifth days. The technique demonstrated here is in sharp contrast to current protocols which usually can take up to one month or more to obtain a positive recombinant adenoviral and baculoviral clone. Therefore, this technique can be used to directly clone a fragment seamlessly into a vector, especially a large one such as adenoviral, baculoviral and BAC plasmid or cosmid, and to modify an existing construct where there are no other available methods.

### Example 2: CRISPR In Vitro Digestion to Remove a DNA Fragment Between Two Recombinase Recognition Sites

### Introduction

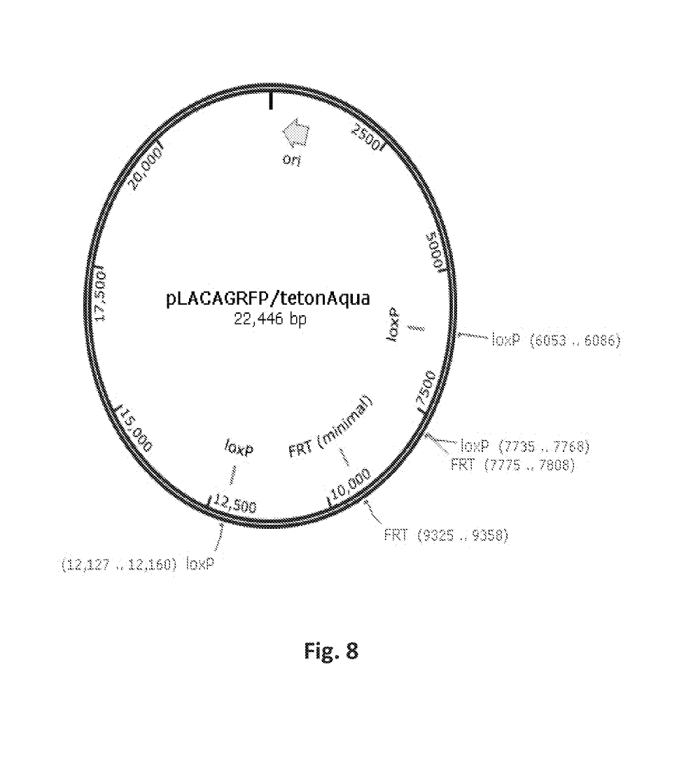

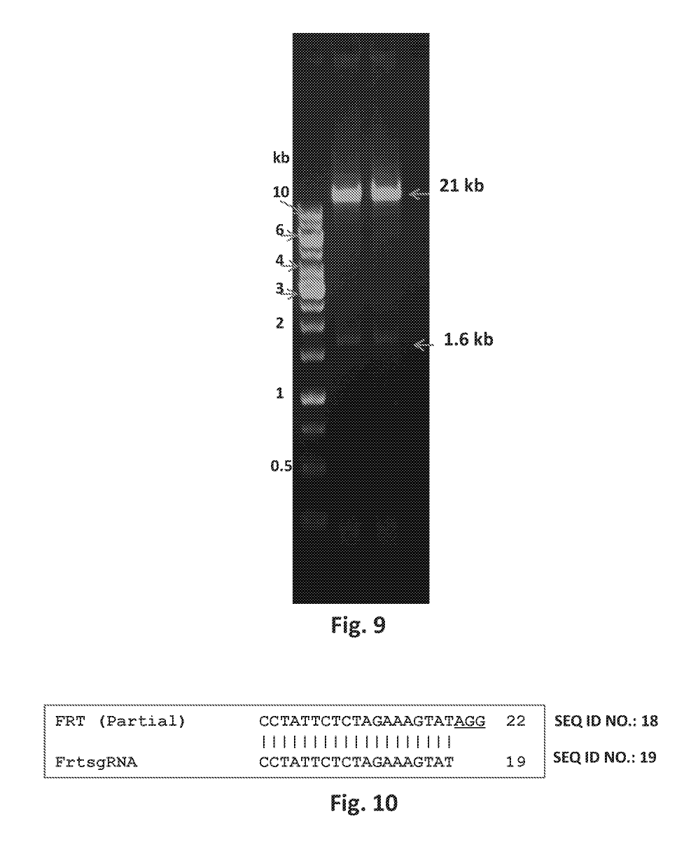

The sequence between two short flippase recognition target (FRT) sites is usually removed by the flippase recombinase in eukaryotic or prokaryotic cells. In this Example, a sgRNA with the CRISPR-RNA (crRNA) complementary to a part of the FRT sequence was synthesized and Cas9/sgRNA digestion of the plasmid pLACAGRFP tetonAqua (FIG. 8) was conducted as aforementioned and discussed elsewhere herein. The results demonstrate that CRISPR in vitro digestion as described herein can be used to remove a DNA fragment between two FRT sites (FIG. 9) with a crRNA sequence complementary to a part of the FRT sequence (FIG. 10). The cleaved plasmid can be ligated to form a new plasmid with the sequence between the two FRT sites removed.

### Example 3: Protospacer Adjacent Motif (PAM)-Independent Cleavage by Cas9/sgRNA Complex In Vitro

CRISPR is an adaptive immune system of bacteria to destroy invading genetic materials. The CRISPR-associated protein-9 (Cas9) is an endonuclease that is guided by a single guide RNA (sgRNA) to the double-stranded target DNA sequence and cleaves the sequence. A sgRNA is composed of a CRISPR-related sequence (crRNA, usually 20-nucleotide), or a protospacer that is from the target sequence and a trans-activating CRISPR-related RNA (tracrRNA) sequence that is from the bacterial CRISPR system. A protospacer adjacent motif (PAM) NGG at the 3′ end of the crRNA sequence in the target sequence can under current CRISPR techniques is needed to facilitate DNA recognition and cleavage by the Cas9/sgRNA complex. 5′-NAG can also have PAM functionality, but the crRNA sequences that use this PAM typically have only about one-fifth cleavage efficiency of those crRNA sequences that use the 5′-NGG. Therefore, the PAM can be represented by 5′-NRG (R=G or A). A PAM sequence at the 3′ end of crRNA sequence can direct the cleavage of the DNA sequence complementary to the crRNA by CRISPR. It is estimated that because of PAM guidance, only about 40% of a genome can be targeted by Cas9. It has been demonstrated that some sequences without a PAM sequence cannot be cleaved by the currently available CRISPR techniques.

Materials and Methods

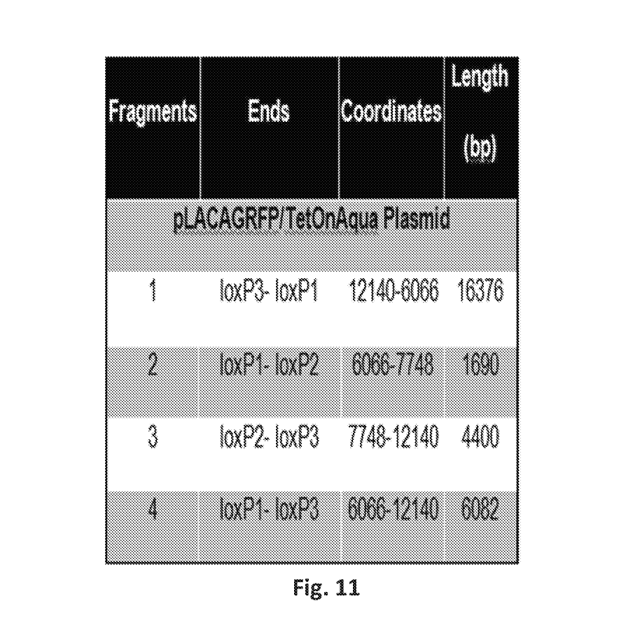

The loxP sequence does not have a NGG PAM sequence. In order to cleave a loxP sequence, a loxPgRNA sequence was chosen (FIG. 10) and synthesized in vitro as described elsewhere within the specification and Examples. The pLrba/CAGRFP/tetonAqua plasmid contains three loxP sites (FIG. 11). The digestion was carried out in an approximately 30 μl reaction mixture containing about 3 μl of 10×Cas9 nuclease reaction buffer, about 30 nM (final concentration) of sgRNA, and about 1 μl of about 1 μM Cas9 nuclease. The final concentration of Cas9 nuclease was about 30 nM. Sterile distilled water was added to bring the total reaction volume to about 30 μl. The mixture was pre-incubated for about 10 min at about 37° C. After pre-incubation, about 1 μl of about 20 mM ATP and about 30 nM substrate DNA with or without about 1 μg of Tth RecA (BioHelix, Beverly, Mass., USA), about 0.5 μg of Thermostable DNA Helicase (BioHelix), about 0.5 μg of Extreme thermostable single-stranded DNA binding protein (ET SSB, BioHelix), or about 10 U of T5 exonuclease was added. The mixture was incubated overnight and an aliquot was analyzed via gel electrophoresis.

Results

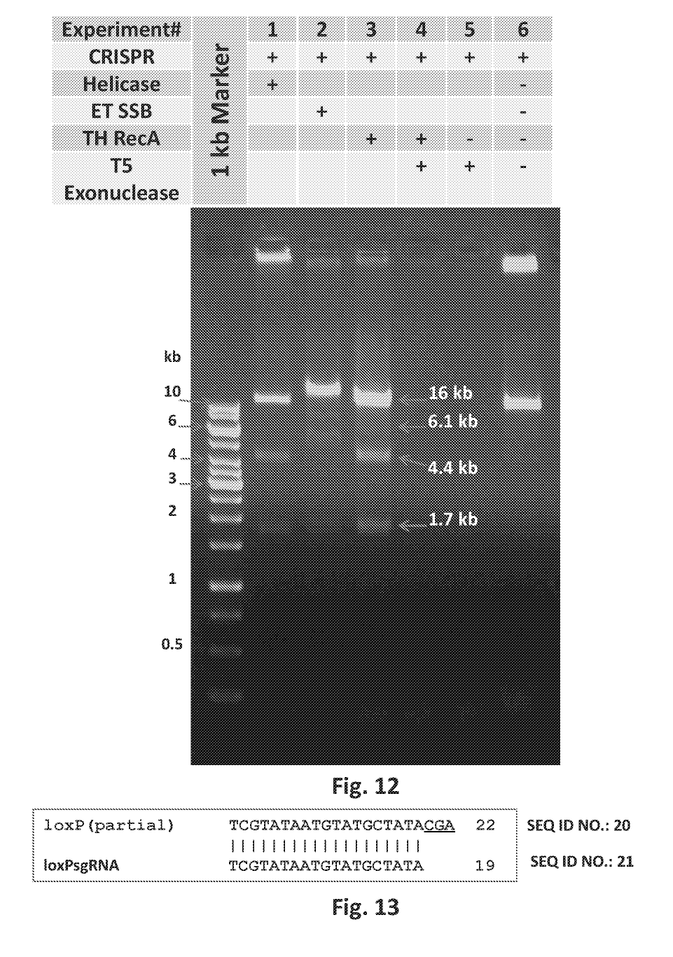

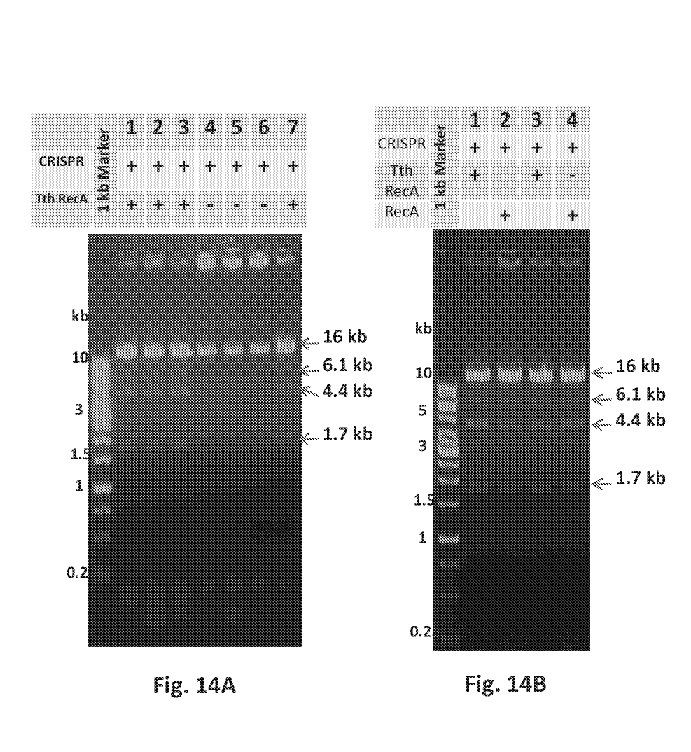

As demonstrated in FIGS. 12-13 and 14A-14B CRISPR enzymes can cleave a sequence without a PAM motif. PAM-independent CRISPR cleavage utilizes proteins that have single-stranded DNA binding property, Tth RecA, Helicase, ET SSB (FIGS. 12-13 and 14A-14B). The substantially complete degradation of plasmid DNA in the CRISPR reaction with T5 exonuclease, which is an endonuclease for single strand DNA, suggests that the Cas9/sgRNA can produce single strand DNA (FIG. 12). The fragments produced by the CRISPR cleavage were as predicted (FIGS. 9, 11, 12, 14A-14B), suggesting that the cleavage was specific. No significant differences were observed between Tth RecA and E. coli RecA on the PAM-independent CRISPR cleavage (FIGS. 14A-14B).

In sum, this Example demonstrates a CRISPR technique that is independent of PAM sequences. As such, this Example demonstrates that the CRISPR targeting sequences are not restricted to those sequences that are located before an NGG sequence. The CRISPR technique demonstrated herein can allow for specific cleavage of any sequence without being limited by the inclusion of a PAM sequence. As such, this technique can allow for up to about 100% of a genome to be targeted by CRISPR.

1. A method comprising:
synthesizing an sgRNA having a crRNA sequence operatively linked to a tracRNA sequence, where the crRNA sequence is complementary to a target sequence in a substrate vector;
incubating the sgRNA with an amount of a Cas9 endonuclease and an amount of the substrate vector to produce a linearized cleaved substrate vector having a cleavage point; and
incubating an amount of linearized cleaved substrate vector with an amount of an insert polynucleotide and an amount of at least one of the following: a DNA ligase, a DNA exonuclease, a DNA polymerase, or a combination thereof,
where the insert polynucleotide comprises a 5′ end sequence that is complementary with a first polynucleotide sequence in the substrate vector and a 3′ end sequence that is complementary with a second polynucleotide sequence in the substrate vector, and
where the first polynucleotide sequence and the second polynucleotide sequence are on opposite sides of the cleavage point.

2. The method of claim 1, wherein the sgRNA is also incubated with an amount of a suitable single stranded DNA binding protein with the amount Cas9 endonuclease during the step of incubating the sgRNA with the amount of a Cas9 endonuclease and the amount of the substrate vector to produce the linearized cleaved substrate vector with the cleavage point.

3. The method of claim 2, wherein the suitable single stranded DNA binding protein is at least one of Tth RecA, a helicase, a single stranded DNA binding protein, or E. coli RecA.

4. The method of claim 3, wherein the sgRNA is also incubated with an amount of a adenosine triphosphate with the amount Cas9 endonuclease and single stranded DNA binding protein during the step of incubating the sgRNA with the amount of a Cas9 endonuclease and the amount of the substrate vector to produce the linearized cleaved substrate vector with the cleavage point.

5. The method of claim 1, wherein the step of synthesizing sgRNA comprises the steps of:
performing a polymerase chain reaction (PCR) to produce a duplex DNA template, wherein the PCR reaction contains an amount of a template DNA, an amount of a forward primer, and an amount of a reverse primer, where the forward primer comprises:
a polynucleotide sequence that can bind a RNA polymerase;
a CRISPR-related RNA (crRNA) polynucleotide, where the crRNA polynucleotide is operatively linked to the polynucleotide sequence that can bind a RNA polymerase; and
a tracrRNA polynucleotide, where the tracrRNA polynucleotide is operatively linked to the crRNA polynucleotide and operatively linked to the polynucleotide sequence that can bind a RNA polymerase; and
performing in vitro transcription on the duplex DNA template to produce the sgRNA.

6. The method of claim 5, wherein the RNA polymerase is T3, T7, or sP6.

7.-8. (canceled)

9. The method of claim 5, wherein the tracrRNA polynucleotide is 19-20 base pairs in length.

10. (canceled)

11. The method of claim 1, wherein the first polynucleotide sequence in the substrate vector and the second polynucleotide sequence in the substrate vector are about 20 to about 40 base pairs in length.

12. The method of claim 1, wherein the ratio of linearized cleaved substrate vector to polynucleotide insert ranges from about 1:1 to about 1:10 to about 10:1.

13. The method of claim 1, wherein incubating an amount of linearized cleaved substrate vector is conducted at about 35° C. to about 50° C.

14. (canceled)

15. The method of claim 1, where the substrate vector is about 2 kb to about 2 Mb.

16. The method of claim 1, wherein the substrate vector is a yeast artificial chromosome, bacterial artificial chromosome, adenoviral vector, cosmid, or baculoviral vector.

17. A method comprising:
synthesizing an sgRNA having a crRNA sequence operatively linked to a tracRNA sequence, where the crRNA sequence is complementary to a target sequence in substrate genomic DNA; and
incubating the sgRNA with an amount of a Cas9 endonuclease, an amount of a suitable single stranded binding protein, and an amount of substrate genomic DNA to produce a cleaved substrate genomic DNA having a cleavage point.

18. The method of claim 17, further comprising:
incubating an amount of cleaved substrate genomic DNA with an amount of an insert polynucleotide and an amount of at least one of the following: a DNA ligase, a DNA exonuclease, a DNA polymerase, or a combination thereof,
where the insert polynucleotide contains a 5′ end sequence that is complementary with a first polynucleotide sequence in the cleaved substrate genomic DNA and a 3′ end sequence that is complementary with a second polynucleotide sequence in the cleaved substrate genomic DNA, and
where the first polynucleotide sequence and the second polynucleotide sequence are on opposite sides of the cleavage point.

19. The method of claim 17, wherein the suitable single stranded DNA binding protein is at least one of Tth RecA, a helicase, Extreme Thermostable single stranded DNA binding protein, E. coli RecA.

20. The method of claim 19, wherein the sgRNA is also incubated with an amount of a adenosine triphosphate with the amount Cas9 endonuclease and single stranded DNA binding protein during the step of incubating the sgRNA with the amount of a Cas9 endonuclease and the amount of the substrate genomic DNA to produce the cleaved substrate genomic DNA having the cleavage point.

21. The method of claim 17, wherein the step of synthesizing sgRNA comprises the steps of:
performing a polymerase chain reaction (PCR) to produce a duplex DNA template, wherein the PCR reaction contains an amount of a template DNA, an amount of a forward primer, and an amount of a reverse primer, where the forward primer comprises:
a polynucleotide sequence that can bind a RNA polymerase;
a CRISPR-related RNA (crRNA) polynucleotide, where the crRNA polynucleotide is operatively linked to the polynucleotide sequence that can bind a RNA polymerase; and
a tracrRNA polynucleotide, where the tracrRNA polynucleotide is operatively linked to the crRNA polynucleotide and operatively linked to the polynucleotide sequence that can bind a RNA polymerase; and
performing in vitro transcription on the duplex DNA template to produce the sgRNA.

22. The method of claim 21, wherein the RNA polymerase is T3, T7, or sP6.

23.-27. (canceled)

28. The method of claim 17, wherein the ratio of linearized cleaved substrate genomic DNA to polynucleotide insert ranges from about 1:1 to about 1:10 to about 10:1.

29. The method of claim 17, wherein incubating an amount of cleaved substrate genomic DNA is conducted at about 35° C. to about 50° C.

30. (canceled)
